(19) United States
(12) Patent Application Publication
(10) Pub. No.: US 2012/0270943 A1
(43) Pub. Date: Oct. 25, 2012

(54) D-aspartic Acid Pro-nutrients with Improved Solubility Characteristics

(76) Inventors: Kyung Suk Kim, Cumming, GA (US); Bryan Joseph Krause, Cumming, GA (US)

(21) Appl. No.: 13/451,751

(22) Filed: Apr. 20, 2012

Related U.S. Application Data
(60) Provisional application No. 61/478,248, filed on Apr. 22, 2011.

(51) Int. Cl. A61K 31/198; A61P 5/24; A61K 31/225; C07C 227/00; C07C 229/24

(52) U.S. Cl. 514/547; 562/571; 560/171; 514/561

(57) ABSTRACT

D-aspartic acid is prepared as its hydrochloride salt or as the hydrochloride salt of its diethyl ester, thereby providing soluble forms of D-aspartic acid that are readily taken up by a human body via its digestive tract. The D-aspartic acid is administered as pills, capsules, powdered drink mixes, beverages, confectionaries, syrups, chewable tablets, nasal sprays, or injectable formulations.

### CROSS-REFERENCE TO RELATED APPLICATIONS

This Non-provisional U.S. patent application claims priority to, and the full benefit of, U.S. Provisional Appl. No. 61/478,248, entitled “D-aspartic acid pro-nutrients with improved solubility characteristics”, filed Apr. 22, 2011, and which is incorporated herein by reference.

### FEDERALLY SPONSORED RESEARCH OR DEVELOPMENT

None

### PARTIES TO A JOINT RESEARCH AGREEMENT

None

### REFERENCE TO A SEQUENCE LISTING

None

### BACKGROUND OF THE INVENTION

1. Technical Field of the Invention

The present invention relates generally to D-aspartic acid pro-nutrients with improved solubility characteristics, and more specifically to D-aspartic acid converted into its hydrochloride salt and D-aspartic acid converted into its diethyl ester hydrochloride salt.

2. Description of Related Art

Light waves vibrate at right angles to their direction of travel. When polarized light (light that is vibrating in only one plane) passes through an optically-active substance, the plane of the light is rotated. Depending on the direction of rotation, the substance rotating the light plane is called either dextrorotatory (for rotation observed by the viewer to be clockwise or in a positive direction) or levorotatory (for counter-clockwise or negative rotation). Optically active materials are not superimposable on their mirror image (asymmetric), while optically inactive materials are (non-asymmetric). To be superimposable, the molecule must be either identical or be able to be made identical by rotation about a single bond.

The mirror images are called enantiomers. All other physical properties other than the rotation of light will be identical for the enantiomers.

The human body typically utilizes L-amino acids and generally does not utilize D-isomers. However, recent studies have shown the possibility of increases in testosterone from ingestion of D-aspartic acid. “The role and molecular mechanism of D-aspartic acid in the release and synthesis of LH and testosterone in humans and rats” Enza Topo1, Andrea Soricelli, Antimo D'Aniellol, Salvatore Ronsini and Gemma D'Aniello, Reproductive Biology and Endocrinology, 7:120, 2009.

Aspartic acid (aminosuccinic acid) has the formula HOOC—CH₂—CH(NH₂)—COOH and is typically formed by nucleophilic addition of ammonia to α,β-unsaturated carbonyl compounds, such as, for exemplary purposes only, from the alpha compound fumaric acid as trans-HOOCCH═CHCOOH+NH₃═HOOC—CH₂—CH(NH₂)—COOH. However, when made by synthetic methods, such as direct ammonolysis or phthalimidomalonic ester method, a racemic mixture (racemate) of aspartic acid (a combination of isomers, and, thus, optically inactive) results, which must be resolved into the optically active materials. More often, stereospecific enzymatic synthesis is utilized to produce optically active materials.

D-aspartic acid is the enantiomer of the dietary amino acid L-aspartic acid. Thus, it is the mirror image of L-aspartic acid. D-aspartic acid occurs naturally in the bodies of animals and is made by the action on a racemic mixture of an enzyme specific for decarboxylation of L-aspartic acid, while leaving D-aspartic acid. D-aspartic acid is known to concentrate most specifically in neuroendocrine tissues such as the pituitary gland, pineal gland, and testes. Recent research has shown the D-aspartic acid serves as a specialized neurotransmitter in parts of the nervous system involved in hormone production. Id.

D-aspartic acid has been shown to stimulate the release of Luteinizing Hormone (LH) and Growth Hormone (GH) from the pituitary gland. Id. Further, it has also been demonstrated to have a direct stimulating effect in the testes upon testosterone production. Id.

Particularly, the above study clearly demonstrated oral administration of D-aspartic acid increases testosterone in humans. Id. This study, done in Italy, showed that doses of almost four (4) grams a day resulted in a rise of testosterone that peaked on day twelve (12) (the last day of the study) at a level 42% greater than day zero. LH levels were also increased significantly. The study used a sample of twenty-three (23) men, with all but three (3) showing significant increases in testosterone. Moreover, the levels of testosterone were still significantly increased three (3) days after discontinuation of the D-aspartic acid. The data in the study strongly indicates that the amino acid builds up in target tissues over time, and then slowly decreases after administration ceases. Id.

The amino acid D-Aspartic Acid is often sold as a dietary supplement. Unfortunately, D-aspartic acid has terrible water solubility (less than 1 gram will dissolve in 220 milliliters of water) which leads to unpredictable bioavailability and the potential for gastrointestinal disturbance. Accordingly, it remains in the digestive tract as a gritty powder and absorbs very slowly and erratically, and a substantial portion of the dose will be excreted in the feces. More importantly, at higher dosages, this powder can cause digestive disturbances in susceptible individuals.

Therefore, it is readily apparent that there is a need for a form of D-aspartic acid that is readily soluble, and which absorbs more readily into the human body.

### BRIEF SUMMARY OF THE INVENTION

This solubility and bioavailability problem is solved by administering D-aspartic acid in the form of its hydrochloride salt or in the form of the hydrochloride salt of its diethyl ester. The preferred embodiment greatly expands the utility of D-aspartic acid for usages as a medicament or a dietary supplement, allowing it to be utilized more easily in beverages, injectable formulations, nasal sprays, and any other form where high aqueous solubility is important.

Briefly described, in a preferred embodiment, the present invention overcomes the above-mentioned disadvantages and meets the recognized need for such a device by providing D-aspartic acid as its hydrochloride salt or as the hydrochloride salt of its diethyl ester, thereby providing soluble forms that can be readily taken up by the body.

According to its major aspects and broadly stated, the present invention in its preferred form is a method of making a soluble form of D-aspartic acid by treating D-aspartic acid with a chlorine-containing material, adding a water miscible solvent, such as, for exemplary purposes only, acetone or ethanol, and precipitating a chlorinated form of the D-aspartic acid, namely, D-aspartic acid hydrochloride. Alternatively, D-aspartic acid diethyl ester hydrochloride may be made by adding D-aspartic acid to stirred ethanol and chlorinating via addition of thionyl chloride.

The D-aspartic acid hydrochloride or D-aspartic acid ethyl ester hydrochloride are administered by ingestion of pills, capsules, powdered drink mixes, beverages, confectionaries, syrups, and/or chewable tablets, or by injection of an aqueous solution, or via a nasal spray. The doses of the D-aspartic acid pro-nutrients are administered in a range from 10 mg to 100 g per day and are provided as a single ingredient or as part of a multi-ingredient regimen.

The administration of D-aspartic acid hydrochloride and/or D-aspartic acid ethyl ester hydrochloride as above has been found to increase testosterone, increase LH and HGH levels, increases strength, muscle mass, endurance and rate of recovery, and improves/increases libido and/or mood.

More specifically, the present invention is D-aspartic acid hydrochloride salt formed by dissolution of D-aspartic acid in hydrochloric acid followed by precipitation by a water miscible organic solvent such as acetone or ethanol. Alternatively, D-aspartic acid diethyl ester hydrochloride is formed by suspending D-aspartic acid in ethanol and adding thionyl chloride, with the resulting solution being dried under vacuum. After an appropriate period of time, D-aspartic acid diethyl ester hydrochloride precipitates from the solution.

Accordingly, a feature and advantage of the present invention is its ability to provide soluble forms of D-aspartic acid.

Another feature and advantage of the present invention is its ability to enhance testosterone production.

Yet a further feature and advantage of the present invention is that it increases luteinizing hormone (LH) and growth hormone (GH or HGH) levels.

Still another feature and advantage of the present invention is its ability to be made synthetically.

Yet another feature and advantage of the present invention is its ability to be administered via a variety of methods.

Yet still another feature and advantage of the present invention is its rapid and predictable absorption characteristics.

Still further features and advantages are that the present invention increases strength, muscle mass, endurance and recovery rate.

Still yet further features and advantages of the present invention are that it improves libido and mood.

These and other features and advantages of the present invention will become more apparent to one skilled in the art from the following description and claims.

### DETAILED DESCRIPTION OF THE PREFERRED EMBODIMENT OF THE INVENTION

In describing the preferred embodiment of the present invention, specific terminology is employed for the sake of clarity. The invention, however, is not intended to be limited to the specific terminology so selected, and it is to be understood that each specific element includes all technical equivalents that operate in a similar manner to accomplish similar functions.

In a first preferred embodiment D-aspartic acid is converted into its hydrochloride salt by dissolution into hydrochloric acid followed by precipitation by a water miscible organic solvent such as acetone or ethanol.

In a second preferred embodiment, D-aspartic acid is converted into its diethyl ester hydrochloride as follows. D-aspartic acid is added to stirred ethanol; subsequently, thionyl chloride is added and the mixture is stirred at room temperature for approximately four (4) hours. A vacuum is applied to remove solvents. Subsequently, the residue is added to ethyl acetate solvent and crystallized, centrifuged and heated.

The D-aspartic acid hydrochloride and D-aspartic acid diethyl ester hydrochloride are administered as a pill, capsule, powdered drink mix, beverage, confectionary, syrup, chewable tablet, nasal spray, or injectable aqueous formulation.

Both D-aspartic acid hydrochloride and D-aspartic acid diethyl ester hydrochloride are subsequently utilized in D-aspartic acid supplement formulations. They are delivered in doses ranging from 10 mg to 100 g per day either as a single ingredient or as part of a multi-ingredient formula or regimen. The high water solubility of D-aspartic acid hydrochloride and D-aspartic acid diethyl ester hydrochloride ensures rapid and predictable absorption characteristics compared to D-aspartic acid alone.

The foregoing description and drawings comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions and the associated drawings. Although specific terms may be employed herein, they are used in a generic and descriptive sense only and not for purposes of limitation. Accordingly, the present invention is not limited to the specific embodiments illustrated herein, but is limited only by the following claims.

What is claimed is:

1. A method of making a soluble form of D-aspartic acid, said method comprising the step of:
making a chlorinated form of said D-aspartic acid.

2. The method of claim 1, wherein said step of making further comprises the step of:
making D-aspartic acid hydrochloride.

3. The method of claim 1, wherein said step of making further comprises the step of:
making D-aspartic acid ethyl ester hydrochloride.

4. D-aspartic acid hydrochloride made by the method of claim 2.

5. D-aspartic acid diethyl ester hydrochloride made by the method of claim 3.

6. The method of claim 2, wherein said method further comprises the step of:
administering said D-aspartic acid hydrochloride to a human in a form selected from the group consisting of pills, capsules, powdered drink mixes, beverages, confectionaries, syrups, chewable tablets, and combinations thereof.

7. The method of claim 2, wherein said method further comprises the step of:
administering said D-aspartic acid hydrochloride to a human as an injectable aqueous formulation.

8. The method of claim 2, wherein said method further comprises the step of:
administering said D-aspartic acid hydrochloride to a human as a nasal spray.

9. The method of claim 3, wherein said method further comprises the step of:
administering said D-aspartic acid diethyl ester hydrochloride to a human in a form selected from the group consisting of pills, capsules, powdered drink mixes, beverages, confectionaries, syrups, chewable tablets, and combinations thereof.

10. The method of claim 3, wherein said method further comprises the step of:
administering said D-aspartic acid diethyl ester hydrochloride to a human as an injectable aqueous formulation.

11. The method of claim 3, wherein said method further comprises the step of:
administering said D-aspartic acid diethyl ester hydrochloride to a human as a nasal spray.

12. A method of delivering D-aspartic acid to a human digestive tract, said method comprising the step of:
ingesting D-aspartic acid pro-nutrients with improved solubility characteristics, wherein said D-aspartic acid pro-nutrients comprise a material selected from the group consisting of D-aspartic acid hydrochloride and D-aspartic acid diethyl ester hydrochloride.

13. The method of claim 12, wherein said step of ingesting D-aspartic acid pro-nutrients with improved solubility characteristics comprises the step of:
ingesting said D-aspartic acid diethyl ester hydrochloride.

14. The method of claim 12, wherein said step of ingesting said D-aspartic acid pro-nutrients with improved solubility characteristics comprises the step of:
ingesting D-aspartic acid hydrochloride.

15. The method of claim 12, wherein said step of ingesting D-aspartic acid pro-nutrients with improved solubility characteristics comprises the step of:
ingesting said D-aspartic acid pro-nutrients in a form selected from the group consisting of pills, capsules, powdered drink mixes, beverages, confectionaries, syrups, chewable tablets, and combinations thereof.

16. The method of claim 12, wherein said step of ingesting D-aspartic acid pro-nutrients with improved solubility characteristics comprises the step of:
ingesting doses of said D-aspartic acid pro-nutrients ranging from 10 mg to 100 g per day.

17. The method of claim 12, wherein said step of ingesting D-aspartic acid pro-nutrients with improved solubility characteristics comprises the step of:
providing said D-aspartic acid pro-nutrients as a single ingredient.

18. The method of claim 12, wherein said step of ingesting D-aspartic acid pro-nutrients with improved solubility characteristics comprises the step of:
providing said D-aspartic acid pro-nutrients as part of a multi-ingredient formula.

19. A method of increasing testosterone, said method comprising the step of:
administering D-aspartic acid pro-nutrients in the amount of 10 mg to 100 g per day.
